(19) United States
(12) Patent Application Publication
(10) Pub. No.: US 2026/0047961 A1
(43) Pub. Date: Feb. 19, 2026

(54) DISPOSABLE PRODUCT APPARATUSES AND METHODS

(71) Applicant: CURT G. JOA, INC., Sheboygan Falls, WI (US)

(72) Inventors: David E. SCHUETTE, Sheboygan Falls, WI (US); Scott A. ROEHRBORN, Sheboygan Falls, WI (US); Cory D. VELDMAN, Sheboygan Falls, WI (US); Christopher A. SCHWARTZ, Sheboygan Falls, WI (US)

(21) Appl. No.: 19/102,983

(22) PCT Filed: Aug. 9, 2023
(86) PCT No.: PCT/US2023/029902

§ 371 Date: Feb. 11, 2025

Related U.S. Application Data
(60) Provisional application No. 63/397,475, filed on Aug. 12, 2022.

(51) Int. Cl. A61F 13/15

(52) U.S. Cl. CPC A61F 13/15617; A61F 13/15682; A61F 13/15723; A61F 13/15764; A61F 13/15804; A61F 2013/1591

(57) ABSTRACT

An apparatus which makes an absorbent insert for an absorbent product includes a die cutting unit which die cuts at least one channel in an assembly that includes an absorbent core material within at least one web. The core material and the at least one web are adhesively secured together. Methods of making same are also provided.

### CROSS-REFERENCE TO RELATED APPLICATIONS

Not applicable

### BACKGROUND OF THE DISCLOSURE

The disclosure herein relates to apparatuses and methods for forming disposable products such as diapers at very high speeds, including forming at least one channel in the disposable products. While the description provided relates to diaper manufacturing, the apparatus and method are easily adaptable to the manufacture of feminine hygiene and adult incontinence products, as well as other applications.

Generally, diapers comprise an absorbent insert or patch and a chassis, which, when the diaper is worn, supports the insert proximate a wearer's body. Additionally, diapers may include other various patches, such as tape tab patches, reusable fasteners and the like. The raw materials used in forming a representative insert are typically cellulose pulp and/or superabsorbent polymers, high internal phase emulsion (HIPE) foams, tissue paper, poly, nonwoven web, acquisition, and elastic, although application specific materials are sometimes utilized. Some of the insert raw materials are provided in roll form and unwound and applied in continuously fed fashion. Other insert raw materials such as superabsorbent polymers and curly fibers, as examples, are provided in non-roll form.

In the creation of a diaper, multiple roll-fed web processes are typically utilized. To create an absorbent insert, the cellulose pulp is unwound from the provided raw material roll and fiberized by a pulp mill. Pulp cores are created using a vacuum forming assembly and placed on a continuous tissue web. Optionally, superabsorbent polymers may be added to the pulp core. In other embodiments, the core may be formed solely of superabsorbent polymers and/or of fibers that are provided to the forming drum from bales. The cores may be formed on a rotary forming system, on a screen, or utilizing other air-laid technologies.

One or multiple nonwoven web layers may be wrapped around the absorbent material with adhesive optionally utilized to provide integrity and stabilization of the core structure. In other cases, the core is formed, unwrapped, on the vacuum forming assembly. Multiple cores, in wrapped or unwrapped form, may be stacked atop one another to form a multiple core assembly. The formed single or multiple core assembly is debulked by proceeding through a calendar unit, which at least partially compresses the core material, thereby increasing its density and structural integrity. After debulking, the core is optionally passed through a segregation or knife unit, where individual inserts are cut. The inserts are conveyed, at the proper pitch, or spacing, to a combining unit where the inserts are enveloped within a diaper chassis.

To improve performance in terms of absorption, comfort and distribution of absorbed liquids, some diapers include longitudinal channels without absorbent material between the two layers of non-woven fabric such as disclosed in WO 2018172902 and EP3403627A1. The methods disclosed in WO 2018172902 and EP3403627A1 provide at least one spreader for spreading the absorbent material on a forming drum which has a suction system that inhibit suction at where the channels are formed so that the absorbent material is not retained at where the channels are formed. The systems that are used to form the channels of WO 2018172902 and EP3403627A1 are complex, and the channels may not be devoid of absorbent material.

Manufacturers would appreciate a simpler system and method for forming channels devoid of absorbent material and which ensures that the resulting diaper achieves the desired performance.

### BRIEF STATEMENT OF THE DISCLOSURE

Provided are apparatuses and methods for forming a channel in a core of a disposable product in web processing operations in a high speed, small footprint environment.

An apparatus which makes an absorbent insert for an absorbent product includes a die cutting unit which die cuts channels in an assembly that includes an absorbent core layer between at least one web layer. The at least one web layer and the absorbent core layer are secured together via adhesive. A method of making same is also provided.

In accordance with one aspect of the invention, an apparatus configured to make an absorbent insert for an absorbent product, the absorbent insert comprising an absorbent core material positioned between at least one sheet. The apparatus includes a core former configured to provide absorbent core material, a first adhesive application unit configured to apply a first adhesive to at least a portion of a form-on web of material, wherein the absorbent core material is bonded to the form-on web by the first adhesive, and a die cutting unit located downstream of the core former and configured to die cut at least one channel that extends through a thickness of at least the absorbent core material.

In embodiments the die cutting unit may include a die and a first roller, the die having a continuous cutting edge which forms the at least one channel and which turns against and in coordination with the first roller.

In embodiments the die cutting unit is configured to die cut at least one channel that extends through a thickness of the absorbent core material and one or more webs.

In embodiments the die cutting unit is configured to die cut at least one channel that extends through a thickness of the absorbent core material. An adhesive applicator, an ultrasonic bonding unit, a thermal bonding unit, or a pressure bonding unit located downstream of the die cutting unit may be provided to bond facing surfaces of a form-on web together or bond facing surfaces of a form-on web and a cover web within the channels.

In some embodiments a debulking unit is configured to debulk the core material. The debulking unit may include a second roller which turns against and in coordination with the first roller. In other embodiments, the debulking unit includes a second roller and a third roller. The debulking unit may be separate from the die cutting unit or combined therewith. A conveyor may be between the debulking unit and the die cutting unit.

In some embodiments the first adhesive application unit does not apply adhesive to a surface of the form-on web where the at least one channel will be die cut.

In embodiments a second adhesive application unit is configured to apply a second adhesive to at least a portion of the form-on web which is folded around the core material. The second adhesive application unit may be configured to apply adhesive across the surface of the cover web where the at least one channel will be die cut or, alternatively, not apply adhesive to a surface of the cover web where the at least one channel will be die cut.

In an aspect of the invention, a method of making an absorbent insert for an absorbent product includes applying adhesive to at least a portion of a form-on web, depositing an absorbent core material onto the adhesive, applying adhesive to at least a portion of a cover web, adhesively securing the cover web to the core material to form an assembly, and die cutting at least one channel into the assembly.

The method may include folding portions of the form-on web around the core material prior to or after adhesively securing the cover web to the core material.

In another aspect of the invention, a method of making an absorbent insert for an absorbent product includes applying adhesive to at least a portion of a form-on web, depositing an absorbent core material onto the adhesive, folding portions of the form-on web around the core material, applying adhesive to the portions of the form-on web, adhesively securing the portion to the core material to form an assembly, and die cutting at least one channel into the assembly.

In some embodiments, the method includes adhesively or mechanical bonding facing layers of the form-on web or the form-on web and the cover web within the at least one channel.

In another aspect of the invention, a method of making an absorbent insert for an absorbent product includes forming an absorbent core material, die cutting at least one channel into the core material, applying adhesive to at least a portion of a form-on web, and depositing the absorbent core material onto the adhesive.

The method may include folding a portion of the form-on web around the core material.

The method may include applying adhesive to at least a portion of a cover web and adhesively securing the cover web to the core material.

In another aspect of the invention, an absorbent insert for an absorbent product includes a first layer of material, a first layer of adhesive on the first layer, and an absorbent core material bonded to the first layer of adhesive, wherein at least the core material has at least one die cut channel formed therein.

In some embodiments the at least one die cut channel is further formed in the first layer of material.

In another aspect of the invention, an absorbent insert for an absorbent product includes a first layer of material, a first layer of adhesive on the first layer, a second layer of material, and an absorbent core material bonded to the first layer of adhesive wherein at least the core material has at least one die cut channel formed therein. The at least one die cut channel is may be formed through the absorbent core material only or also formed through one or both of the first and second layers of material. Some embodiments include a second layer of adhesive on the second layer.

### BRIEF DESCRIPTION OF THE DRAWINGS

The drawings illustrate embodiments presently contemplated for carrying out the disclosure.

In the drawings:

FIG. 1A is a schematic of a first embodiment of a representative web processing apparatus;

FIG. 1B is a schematic of an alternate embodiment of the representative web processing apparatus;

FIG. 2 is a top plan view of inserts formed by the web processing apparatus of FIGS. 1A and 1B;

FIG. 3 is a schematic representation of a cross-section of an insert formed by the web processing apparatus of FIGS. 1A and 1B, shown along line 3-3 of FIG. 3;

FIG. 4A is a first schematic representation of a cross-section of an assembly prior to die cutting which is formed in the web processing apparatus of FIGS. 1A and 1B;

FIG. 4B is a second schematic representation of a cross-section of an assembly prior to die cutting which is formed in the web processing apparatus of FIGS. 1A and 1B;

FIG. 5 is a third schematic representation of a cross-section of an assembly prior to die cutting which is formed in an alternative embodiment of the web processing apparatus of FIGS. 1A and 1B;

FIG. 6 is a schematic of a second embodiment of a representative web processing apparatus;

FIG. 7 is a top plan view of inserts formed by the web processing apparatus of FIG. 6;

FIG. 8 is a schematic representation of a cross-section of an insert formed by the web processing apparatus of FIG. 6, shown along line 7-7 of FIG. 7;

FIG. 9A is a first schematic representation of a cross-section of an assembly prior to die cutting which is formed in the web processing apparatus of FIG. 6;

FIG. 9B is a second schematic representation of a cross-section of an assembly prior to die cutting which is formed in the web processing apparatus of FIG. 6;

FIG. 10 is a third schematic representation of a cross-section of an assembly prior to die cutting which is formed in an alternative embodiment of the web processing apparatus of FIG. 6;

FIG. 11 is a schematic of a third embodiment of a representative web processing apparatus;

FIG. 12 is a top plan view of inserts formed by the web processing apparatus of FIG. 11;

FIG. 13 is a schematic representation of a cross-section of an insert formed by the web processing apparatus of FIG. 11, shown along line 13-13 of FIG. 12;

FIG. 14A is a first schematic representation of a cross-section of an assembly prior to die cutting which is formed in the web processing apparatus of FIG. 11;

FIG. 14B is a second schematic representation of a cross-section of an assembly prior to die cutting which is formed in the web processing apparatus of FIG. 11;

FIG. 15 is a third schematic representation of a cross-section of an assembly prior to die cutting which is formed in an alternative embodiment of the web processing apparatus of FIG. 11;

FIG. 16 is a schematic of a fourth embodiment of a representative web processing apparatus;

FIG. 17 is a top plan view of inserts formed by the web processing apparatus of FIG. 16;

FIG. 18 is a schematic representation of a cross-section of an insert formed by the web processing apparatus of FIG. 16, shown along line 18-18 of FIG. 17;

FIG. 19A is a first schematic representation of a cross-section of an assembly prior to die cutting which is formed in the web processing apparatus of FIG. 16;

FIG. 19B is a second schematic representation of a cross-section of an assembly prior to die cutting which is formed in the web processing apparatus of FIG. 16;

FIG. 20 is a third schematic representation of a cross-section of an assembly prior to die cutting which is formed in an alternative embodiment of the web processing apparatus of FIG. 16;

FIG. 21 is a schematic of a fifth embodiment of a representative web processing apparatus;

FIG. 22 is a top plan view of inserts formed by the web processing apparatus of FIG. 21;

FIG. 23 is a schematic representation of a cross-section of an insert formed by the web processing apparatus of FIG. 22, shown along line 23-23 of FIG. 22;

FIG. 24A is a first schematic representation of a cross-section of an assembly prior to die cutting which is formed in the web processing apparatus of FIG. 21;

FIG. 24B is a second schematic representation of a cross-section of an assembly prior to die cutting which is formed in the web processing apparatus of FIG. 21;

FIG. 25 is a third schematic representation of a cross-section of an assembly prior to die cutting which is formed in an alternative embodiment of the web processing apparatus of FIG. 21;

FIG. 26A is a schematic of a sixth embodiment of a representative web processing apparatus;

FIG. 26B is a schematic of an alternate sixth embodiment of a representative web processing apparatus;

FIG. 27 is a top plan view of inserts formed by the web processing apparatus of FIGS. 26A and 26B;

FIG. 28 is a schematic representation of a cross-section of an insert formed by the web processing apparatus of FIGS. 26A and 26B, shown along line 28-28 of FIG. 27;

FIG. 29A is a first schematic representation of a cross-section of a core prior to die cutting which is formed in the web processing apparatus of FIGS. 26A and 26B;

FIG. 29B is a second schematic representation of a cross-section of the core after die cutting which is formed in the web processing apparatus of FIGS. 26A and 26B;

FIG. 30A is a schematic of a seventh embodiment of a representative web processing apparatus;

FIG. 30B is a schematic of an alternate seventh embodiment of the representative web processing apparatus;

FIG. 31 is a top plan view of inserts formed by the web processing apparatus of FIGS. 30A and 30B;

FIG. 32 is a schematic representation of a cross-section of an insert formed by the web processing apparatus of FIGS. 30A and 30B, shown along line 32-32 of FIG. 31;

FIG. 33A is a first schematic representation of a cross-section of a core prior to die cutting which is formed in the web processing apparatus of FIGS. 30A and 30B; and

FIG. 33B is a second schematic representation of a cross-section of the core after die cutting which is formed in the web processing apparatus of FIGS. 30A and 30B.

### DETAILED DESCRIPTION

While the present disclosure may be susceptible to embodiment in different forms, there is shown in the Figures, and will be described herein in detail, specific embodiments, with the understanding that the present disclosure is to be considered an exemplification of the principles of the present disclosure, and is not intended to limit the present disclosure to that as illustrated.

As such, references to a feature or aspect are intended to describe a feature or aspect of an example of the present disclosure, not to imply that every embodiment thereof must have the described feature or aspect. Furthermore, it should be noted that the description illustrates a number of features. While certain features have been combined together to illustrate potential system designs, those features may also be used in other combinations not expressly disclosed. Thus, the depicted combinations are not intended to be limiting, unless otherwise noted.

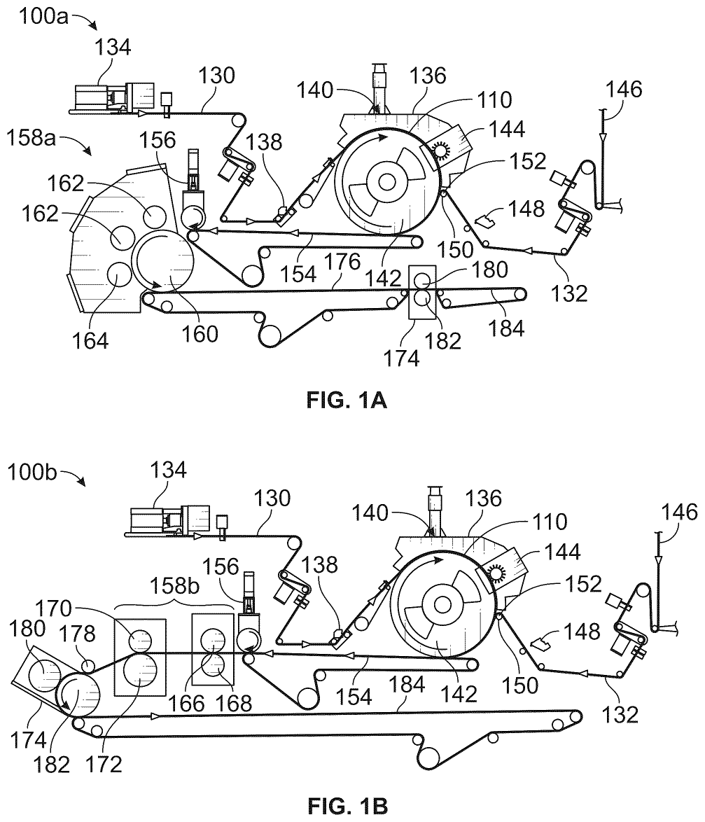

It is noted that the present apparatuses and methods are described herein with respect to disposable products such as diapers, but as previously mentioned, can be applied to a wide variety of processes in which discrete components are applied sequentially. Embodiments of an apparatus 100a, 100b, 200, 300, 400, 500, 600a, 600b, 700a, 700b, shown in FIGS. 1A, 1B, 6, 11, 16, 21, 26A, 26B, 30A, 30B that form an insert 102, 202, 302, 402, 502, 602, 702 for a disposable product are disclosed. The insert 102, 202, 302, 402, 502, 602, 702 includes at least one channel 104, 204, 304, 404, 504, 604, 704 which is formed by die cutting. The die cutting provides a simple solution to formation of the at least one channel 104, 204, 304, 404, 504, 604, 704 in the insert 102, 202, 302, 402, 502, 602, 702, cleanly forms the at least one channel 104, 204, 304, 404, 504, 604, 704 in the insert 102, 202, 302, 402, 502, 602, 702, and forms a consistent channel shape.

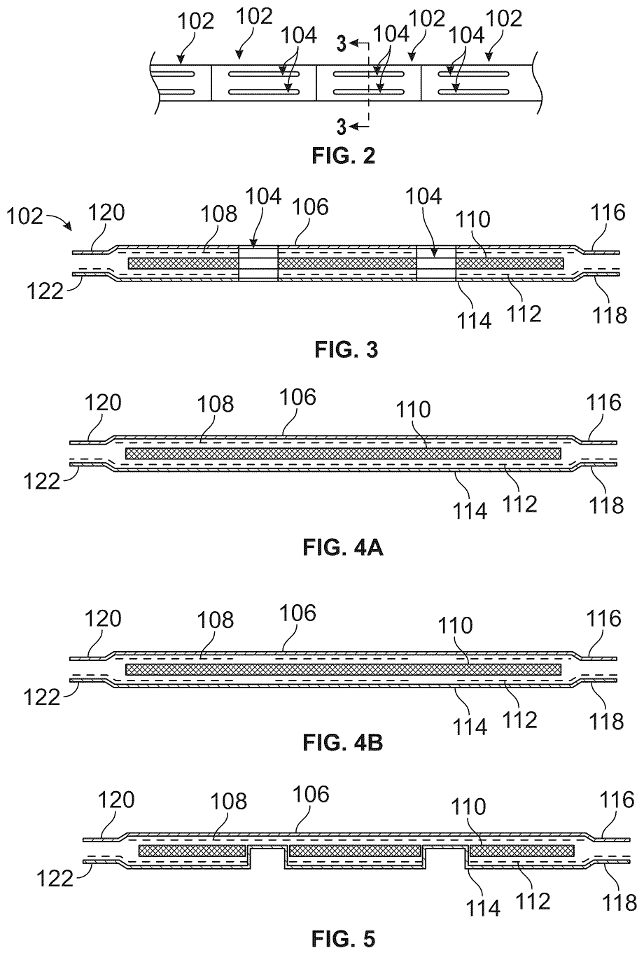

In a first embodiment as shown in FIGS. 2 and 3, the insert 102 includes a first sheet 106 formed of a web material such as nonwoven material, tissue paper, or acquisition distribution layer (ADL), as non-limiting examples, a first adhesive layer 108, a layer of absorbent core material 110, a second adhesive layer 112, and a second sheet 114 formed of a web material such as nonwoven material, tissue paper, or ADL, as non-limiting examples. The first adhesive layer 108 is provided between the first sheet 106 and the core material layer 110. The second adhesive layer 112 is provided between the core material layer 110 and the second sheet 114. First longitudinally extending edge portions 116, 118 of the respective sheets 106, 114 are adhesively secured to each other by the second adhesive layer 112, and second longitudinally extending edge portions 120, 122 of the respective sheets 106, 114 are adhesively secured to each other by the second adhesive layer 112. The channel(s) 104 extend through the first sheet 106, the first adhesive layer 108, the core material layer 110, the second adhesive layer 112, and the second sheet 114. The spacing shown in FIG. 3 is for illustrative purposes only to show the different layers.

In a second embodiment as shown in FIGS. 7 and 8, the insert 202 includes a first sheet 206 formed of a web material such as nonwoven material, tissue paper, or ADL, as non-limiting examples, a first adhesive layer 208, an absorbent core material layer 210, a second adhesive layer 212, and a second sheet 214 formed of a web material such as nonwoven material, tissue paper, or ADL, as non-limiting examples. The first adhesive layer 208 is provided between the first sheet 206 and the core material layer 210. Longitudinally extending edge portions 216, 220 of the first sheet 206 are folded around side edges of the core material layer 210 and folded over sections 222, 224 abut against a second surface of the core material layer 210. The second adhesive layer 212 is between the second sheet 214 and the core material layer 210, and is between the second sheet 214 and the folded over sections 222, 224 of the longitudinally extending edge portions 216, 220. The channel(s) 204 extend through the first sheet 206, the first adhesive layer 208, the core material layer 210, the second adhesive layer 212, and the second sheet 214. The spacing shown in FIG. 8 is for illustrative purposes only to show the different layers.

In a third embodiment as shown in FIGS. 12 and 13, the insert 302 includes a first sheet 306 formed of a web material such as nonwoven material, tissue paper, or ADL, as non-limiting examples, a first adhesive layer 308, an absorbent core material layer 310, a second adhesive layer 312, a second sheet 314 formed of a web material such as nonwoven material, tissue paper, or ADL, as non-limiting examples, and a third adhesive layer 326. The first adhesive layer 308 is provided between the first sheet 306 and the core material layer 310. The second adhesive layer 312 is between the second sheet 314 and the core material layer 310. Longitudinally extending edge portions 316, 320 of the sheet 306 are folded around side edges of the core material layer 310 and folded over sections 322, 324 are proximate to a second surface of the core material layer 310 and proximate to a second surface of the second sheet 314. The third adhesive layer 326 is between the second sheet 314 and the folded over sections 322, 324. The channel(s) 304 extend through the first sheet 306, the first adhesive layer 308, the core material layer 310, the second adhesive layer 312, and the second sheet 314. The spacing shown in FIG. 13 is for illustrative purposes only to show the different layers.

In a fourth embodiment as shown in FIGS. 17 and 18, the insert 402 includes a sheet 406 formed of a web material such as nonwoven material, tissue paper, or ADL, as non-limiting examples, a first adhesive layer 408, an absorbent core material layer 410, and a second adhesive layer 410. The first adhesive layer 408 is provided between the sheet 406 and the core material layer 410. Longitudinally extending edge portion 416 of the sheet 406 is folded around a side edge of the core material layer 410 and a folded over section 422 abuts against a second surface of the core material layer 410. Longitudinally extending edge portion 420 of the sheet 406 is folded around an opposite side edge of the core material layer 410 and a folded over section 424 is proximate to the second surface of the core material layer 410 and overlaps the folded over section 422. The second adhesive layer 410 is between the folded over sections 422, 424. The channel(s) 404 extend through the first sheet 406, the first adhesive layer 408, the core material layer 410, and edge portion 420 and/or 424. The spacing shown in FIG. 18 is for illustrative purposes only to show the different layers.

In a fifth embodiment as shown in FIGS. 22 and 23, the insert 502 includes a sheet 506 formed of a web material such as nonwoven material, tissue paper, or ADL, as non-limiting examples, a first adhesive layer 508, an absorbent core material layer 510, a second adhesive layer 510, and a third adhesive layer 528. The first adhesive layer 508 is provided between the sheet 506 and the core material layer 510. The second adhesive layer 512 is provided on the opposite side of the core material layer 510. Longitudinally extending edge portion 516 of the sheet 506 is folded around a side edge of the core material layer 510 and a folded over section 522 abuts against a first portion of the second adhesive layer 512. Longitudinally extending edge portion 520 of the sheet 506 is folded around an opposite side edge of the core material layer 510 and a folded over section 524 abuts against a second portion of the second adhesive layer 512 and overlaps the folded over section 522. The third adhesive layer 528 is between the folded over sections 522, 524. The channel(s) 504 extend through the first sheet 506, the first adhesive layer 508, the core material layer 510, the second adhesive layer 512, and edge portion 516 and/or 524. The spacing shown in FIG. 23 is for illustrative purposes only to show the different layers.

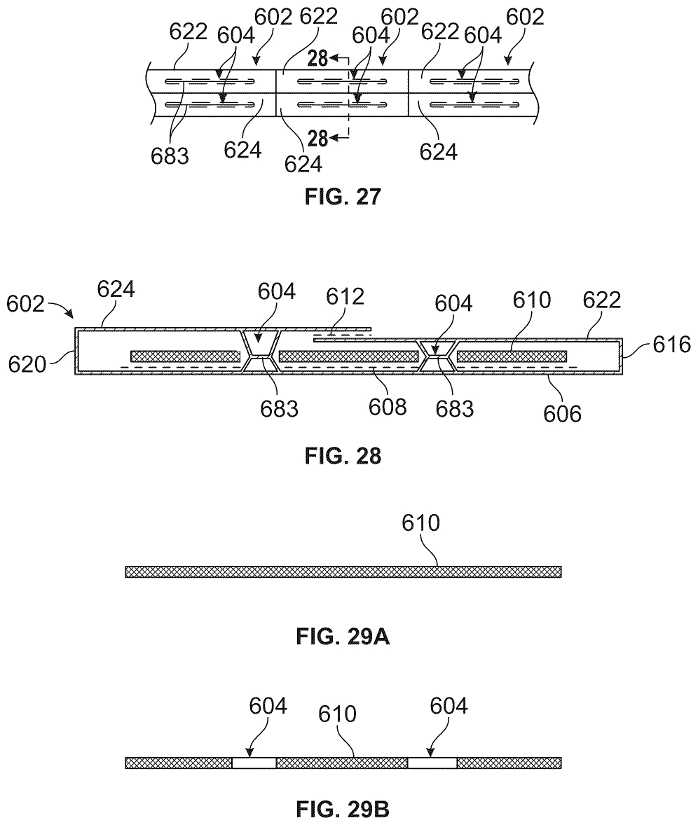

In a sixth embodiment as shown in FIGS. 27 and 28, the insert 602 includes a sheet 606 formed of a web material such as nonwoven material, tissue paper, or ADL, as non-limiting examples, a first adhesive layer 608, an absorbent core material layer 610, and a second adhesive layer 612. The first adhesive layer 608 is provided between the sheet 606 and the core material layer 610. Longitudinally extending edge portion 616 of the sheet 606 is folded around a side edge of the core material layer 610 and a folded over section 622 abuts against a second surface of the core material layer 610. Longitudinally extending edge portion 620 of the sheet 606 is folded around an opposite side edge of the core material layer 610 and a folded over section 624 is proximate to the second surface of the core material layer 610 and overlaps the folded over section 622. The second adhesive layer 612 is between the folded over sections 624, 622. The channel(s) 604 only extend through the core material layer 510. The spacing shown in FIG. 28 is for illustrative purposes only to show the different layers.

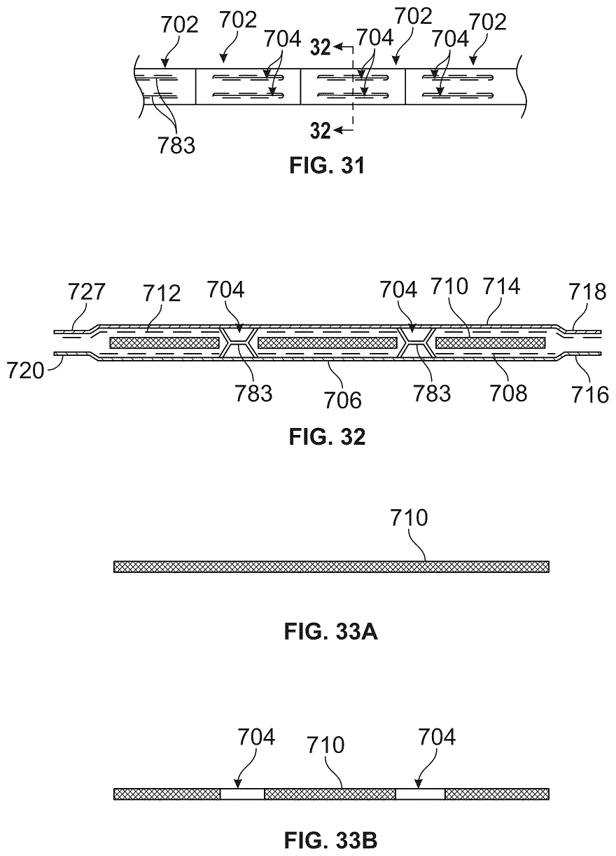

In a seventh embodiment as shown in FIGS. 31 and 32, the insert 702 includes a first sheet 706 formed of a web material such as nonwoven material, tissue paper, or acquisition distribution layer (ADL), as non-limiting examples, a first adhesive layer 708, a layer of absorbent core material 710, a second adhesive layer 712, and a second sheet 714 formed of a web material such as nonwoven material, tissue paper, or ADL, as non-limiting examples. The first adhesive layer 708 is provided between the first sheet 706 and the core material layer 710. The second adhesive layer 712 is provided between the core material layer 710 and the second sheet 714. First longitudinally extending edge portions 716, 718 of the respective sheets 706, 714 are adhesively secured to each other by the second adhesive layer 712, and second longitudinally extending edge portions 720, 722 of the respective sheets 706, 714 are adhesively secured to each other by the second adhesive layer 712. The channel(s) 704 extend through the core material layer 710. The spacing shown in FIG. 32 is for illustrative purposes only to show the different layers.

At least one channel 104, 204, 304, 404, 504, 604, 704 is provided in the insert 102, 202, 302, 402, 502, 602, 702 and extends in a longitudinal direction of the insert 102, 202, 302, 402, 502, 602, 702. As shown, two laterally spaced channels 104, 204, 304, 404, 504, 604, 704 are provided in each insert 102, 202, 302, 402, 502, 602, 702, but one or more channels 104, 204, 304, 404, 504, 604, 704 may be provided. The channels 104, 204, 304, 404, 504, 604, 704 can align with each other in each insert 102, 202, 302, 402, 502, 602, 702, or can be offset from each other. The channels 104, 204, 304, 404, 504, 604, 704 may be a variety of shapes, such as for example a rectangular shape, a peanut shape, curved lines, dots, etc., and the channels 104, 204, 304, 404, 504, 604, 704 in each insert 102, 202, 302, 402, 502, 602, 702 may differ in shape. The channels 104, 204, 304, 404, 504, 604, 704 may be closed ended (such that core material layer 110, 210, 310, 410, 510, 610, 710 is provided at the front longitudinal end of the channel 104, 204, 304, 404, 504, 604, 704 and is provided at the rear longitudinal end of the channel 104, 204, 304, 404, 504, 604, 704), or the channels 104, 204, 304, 404, 504, 604, 704 may be open ended (such that core material layer 110, 210, 310, 410, 510, 610, 710 is not provided at the front longitudinal end of the channel 104, 204, 304, 404, 504, 604, 704 and is not provided at the rear longitudinal end of the channel 104, 204, 304, 404, 504, 604, 704).

The embodiments of the apparatus 100a, 100b which form the first embodiment of the insert 102 are shown in FIGS. 1A and 1B. A first, form-on web 130 which will form the first sheet 106 of the insert 102 is provided and has a planar first surface and an opposite planar second surface, and a second, cover web 132 which will form the second sheet 114 of the insert 102 is provided and has a planar first surface and an opposite planar second surface. Each web 130, 132 defines a longitudinal axis which is parallel to the direction of movement of each web 130, 132 through the apparatus 100a, 100b and a lateral axis which is transverse to the direction of movement of each web 130, 132 through the apparatus 100a, 100b.

The form-on web 130 is continuously fed from a source 134, such as a roll of material, to a core former 136. The form-on web 130 may be fed around a series of rollers as it proceeds from the source 134 to the core former 136. A first adhesive application station 138 is provided between the source 134 and the core former 136 and applies the adhesive layer 108 to at least a portion of the second surface of the form-on web 130. Adhesive is not applied to first and second longitudinally extending edge portions 116, 120 of the second surface of the form-on web 130. The adhesive layer 108 may be applied to the entire second surface of the form-on web 130 between the first and second longitudinally extending edge portions 116, 120 as shown in FIG. 4A, or may be applied to the second surface of the form-on web 130 between the first and second longitudinally extending edge portions 116, 120 except to the sections where the channels 104 will be formed as shown in FIG. 4B.

A super absorbent polymer (SAP)/pulp mixture, or pulp/SAP blend, or pulp only, or SAP only which will form the core material layer 110 is fed onto the core former 136. In the illustrated embodiment, the form-on web 130, having the adhesive layer 108 applied thereto, passes between an outlet of the forming duct 140 of the core former 136 and a cylindrical rotating vacuum drum 142 of the core former 136. As the form-on web 130 passes by the outlet, the core material layer 110 is deposited onto the adhesive layer 108 on the form-on web 130. The vacuum of the vacuum drum 142 is drawn through holes in the vacuum drum 142 and holds the form-on web 130 and core material layer 110 against the vacuum drum 142. The vacuum drum 142 may include a continuous three-dimensional fluff receiving pocket or depression or a plurality of discrete, three-dimensional fluff receiving pockets or depressions on an outer circumferential surface thereof. The core former 136 may include a scarf device 144 downstream of the outlet which scarfs the core material layer 110 to level the top thereof. This forms a first assembly. While core former 136 is illustrated as a rotary forming system, alternative embodiments may utilize a screen former (not shown) that deposits the core material layer 110 in one or more stages onto the form-on web 130 while the form-on web 130 travels on a flat conveyor.

The cover web 132 is continuously fed from a source 134, such as a roll of material, to the exit of the core former 136. The cover web 132 may be fed around a series of rollers as it proceeds from the source 134 to the exit of the core former 136. A second adhesive application station 148 is provided between the source 134 and the core former 136 and applies the second adhesive layer 112 to the first surface of the cover web 132. The second adhesive layer 112 may be applied to the entire first surface of the cover web 132 including the first and second longitudinally extending edge portions 118, 122 as shown in FIG. 4A, or may be applied to the second surface of the form-on web 130 except to the sections where the channels 104 will be formed as shown in FIG. 4B.

The first assembly and the cover web 132, having the second adhesive layer 112 applied thereto, passes through a combining station 150 which is downstream of the core former 136. In the embodiment as shown, the combining station 150 includes the drum 142 and a nip roller 152. The nip roller 152 rotates in coordination with the drum 142. Longitudinally extending edge portions 116, 118 face each other, and longitudinally extending edge portions 120, 122 face each other. The first assembly and the cover web 132 pass through the nip formed between the drum 142 and the nip roller 152 and this bonds the second adhesive layer 112 to the core material layer 110. This forms a second assembly.

The second assembly then passes between the drum 142 and a conveyor 154, and the second assembly translates along the conveyor 154 to an edge bonding unit 156 which is downstream of the combining station 150. The edge bonding unit 156 adheres the first longitudinally extending edge portions 116, 118 together by the second adhesive layer 112 and adheres the second longitudinally extending edge portions 120, 122 together by the second adhesive layer 112. The edge bonding unit 156 may be movable roller(s) that press down onto the conveyor 154. This forms a third assembly.

Thereafter, the third assembly passes through a debulking and channel cutting station 158a, 158b which is downstream of the edge bonding unit 156. The embodiment of FIG. 1A shows a first example of the debulking and channel cutting station 158a, and the embodiment of FIG. 1B shows a second example of the debulking and channel cutting station 158b.

As shown in the embodiment of the apparatus 100a of FIG. 1A, the debulking and channel cutting station 158a includes a cylindrical rotating anvil roller 160, at least one rotating calendaring drum 162 (shown as two calendaring drums in FIG. 1A) and a rotating channel die drum 164. The calendaring drum(s) 162 rotates in coordination with the anvil roller 160, and the die drum 164 turns against and rotates in coordination with the anvil roller 160. The third assembly passes through the nip formed between the calendaring drum(s) 162 and the anvil roller 160 to debulk the third assembly, which at least partially compresses the core material layer 110, thereby increasing its density and structural integrity. In some embodiments, vacuum is drawn through holes in the anvil roller 160 and holds the third assembly as the third assembly passes through the debulking and channel cutting station 158a. In other embodiments, anvil roller 160 may operate without vacuum. After debulking, the third assembly passes between the channel die drum 164 and the anvil roller 160. The channel die drum 164 has a continuous cutting edge formed in the desired shape of the channel 104. The channel die drum 164 turns against and in coordination with the anvil roller 160. In some embodiments, the channel die drum 164 includes vacuum in at least the channel areas to transport the trimmed material away from the insert assembly for further processing and/or recycling. Alternatively, a separate vacuum unit (not shown) may be provided in close proximity to the channel die drum 164 to collect and transport the trimmed material from the channels. As the third assembly passes between the channel die drum 164 and the anvil roller 160, the channel(s) 104 are die cut. When the third assembly is die cut to form the channels 104 therein, the thickness of the form-on and cover webs 130, 132 and the core material layer 110 are cut completely through from the first surface of the form-on web 130 to the second surface of the cover web 132. This forms a fourth assembly.

As shown in the embodiment of the apparatus 100b of FIG. 1B, the debulking and channel cutting station 158b includes at least one debulking unit and a separate die cutting unit. The debulking and channel cutting station 158b of the embodiment of FIG. 1B does not use a single anvil roller as provided for in the embodiment of FIG. 1A. The debulking unit(s) includes a rotating calendaring drum 166 and a rotating anvil roller 168. The calendaring drum 166 turns in coordination with the anvil roller 168. The third assembly passes between the nip formed by the calendaring drum 166 and anvil roller 168 (or drums if more than one debulking unit is provided) to debulk the third assembly, which at least partially compresses the core material layer 110, thereby increasing its density and structural integrity. The die cutting unit includes a rotating channel die drum 170 and a rotating anvil roller 172. The channel die drum 170 turns against and in coordination with the anvil roller 172. The channel die drum 170 has at continuous cutting edge formed in the desired shape of the channel 104. After debulking, the third assembly passes between the channel die drum 170 and the anvil roller 172 and the channel(s) 104 are die cut. When the third assembly is die cut to form the channels 104 therein, the thickness of the form-on and cover webs 130, 132 and the core material layer 110 are cut completely through from the first surface of the form-on web 130 to the second surface of the cover web 132. This forms a fourth assembly. The provision of separate debulking unit and die cutting unit allows for the easy replacement of the die cutting unit when the channel die drum 170 and/or the anvil roller 172 become worn during use.

The fourth assembly exits the debulking and channel cutting station 158a, 158b and is conveyed to a cutting unit 174 which is downstream of the debulking and channel cutting station 158a, 158b. As shown in the embodiment of FIG. 1A, the fourth assembly is translated to the cutting unit 174 by a conveyor 176. As shown in the embodiment of FIG. 1B, the fourth assembly is translated to the cutting unit 174 by a nip roller 178 that turns with the cutting unit 174. The cutting unit 174 severs the fourth assembly into individual inserts 102. In an embodiment, the cutting unit 174 includes a roller 180 having one or more knives thereon and an anvil roller 182. When the fourth assembly passes between the rotating roller 180 and the anvil roller 182, the fourth assembly is cut in the transverse direction to form the individual inserts 102. Thereafter, the individual inserts 102 are transported for example by a conveyor 184 to another apparatus (not shown) for completion of the disposable product in which the inserts 102 are encapsulated between a back sheet formed of an impermeable material and at least one hydrophilic top sheet. Other layers may be provided in the final disposable product.

Adhesive layers 108 and 112 may be applied using spray coating or slot coating techniques. In the embodiment shown in FIG. 4B, since the adhesive layers 108, 112 are not applied to the webs 130, 132 where the channel(s) 104 will eventually be die cut by the channel die drum 164, 170, this assists in preventing the contamination of the channel die drum 164, 170 by adhesive.

FIG. 5 shows an alternative cross-section of an embodiment before the die cutting takes place. In the embodiments of the apparatus 100a, 100b used to form the embodiments of FIGS. 4A and 4B, the rotating drum 142 of the core former 136 has a smooth outer surface which forms a generally uniform thickness of the core material layer 110 on the form-on web 130. The rotating drum 142 of the core former 136 can have structure, for example insert(s) or projection(s) located in its forming surface that inhibit suction at select portions on the forming surface to lessen the thickness of the core material layer 110 deposited on the form-on web 130, or to prevent the core material layer 110 from accumulating on the form-on web 130 above those structures. When no core material layer 110 is deposited on the form-on web 130 as shown in FIG. 5, when the third assembly is die cut to form the channels 104 therein, only the thickness of the form-on and cover webs 130, 132 are die cut.

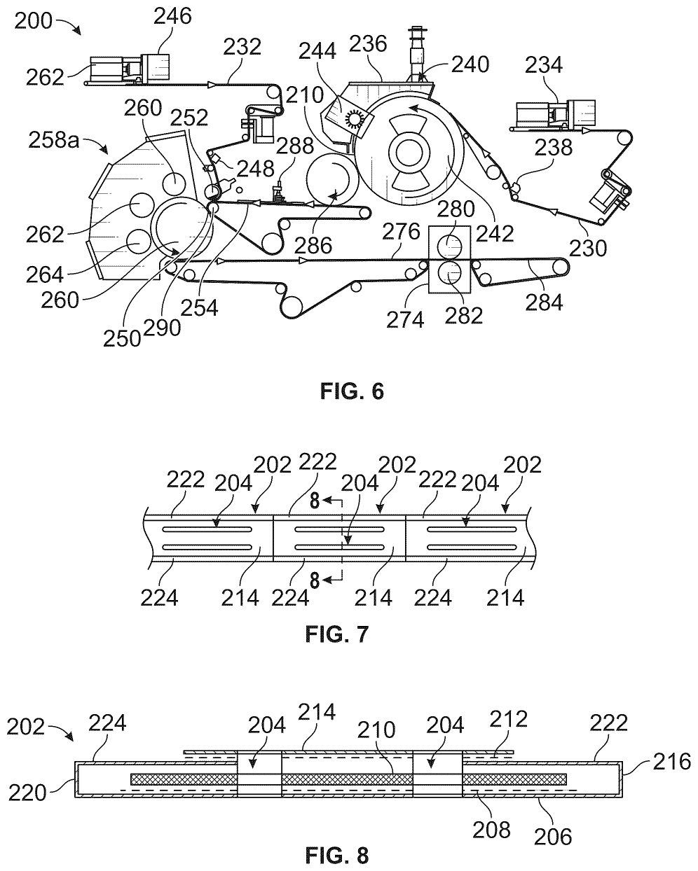

An embodiment of the apparatus 200 which forms the second embodiment of the insert 202 is shown in FIG. 6. Like elements of this embodiment to those of the first embodiment are denoted with like reference numerals in the two hundreds.

The first assembly is formed in an identical manner, and therefore the specifics are not repeated herein. The first assembly exits the core former 236 and may pass around a roller 286 that rotates with the vacuum drum 242 of the core former 236 and the conveyor 254. The first assembly is transported along the conveyor 254 to a folding unit 288, which may be a folding board, plate, rods, rollers, plow, air curtain or other known folding technologies, which is downstream of the core former 236. The longitudinally extending edge portions 216, 220 are folded over around the side edges of the core material layer 210 and onto the second surface of the core material layer 210 by the folding unit 288 to form the folded over sections 222, 224. The folded over sections 222, 224 partially overlap the second surface of the core material layer 210 such that a central portion of the second surface of the core material layer 210 is not covered by the folded over sections 222, 224. In an optional step, an adhesive application station (not show) may be provided between the roller 286 and the folding unit 288 to apply an adhesive layer to the first surface of the longitudinally extending edge portions 216, 220 to bond the longitudinally extending edge portions 216, 220 to the core material layer 210 when folded around the core material layer 210. This forms a second assembly.

Thereafter, the second assembly is transported to the combining station 250 by the conveyor 254. In the embodiment as shown, the combining station 250 includes the nip roller 252 that presses against a roller 290 of the conveyor 254.

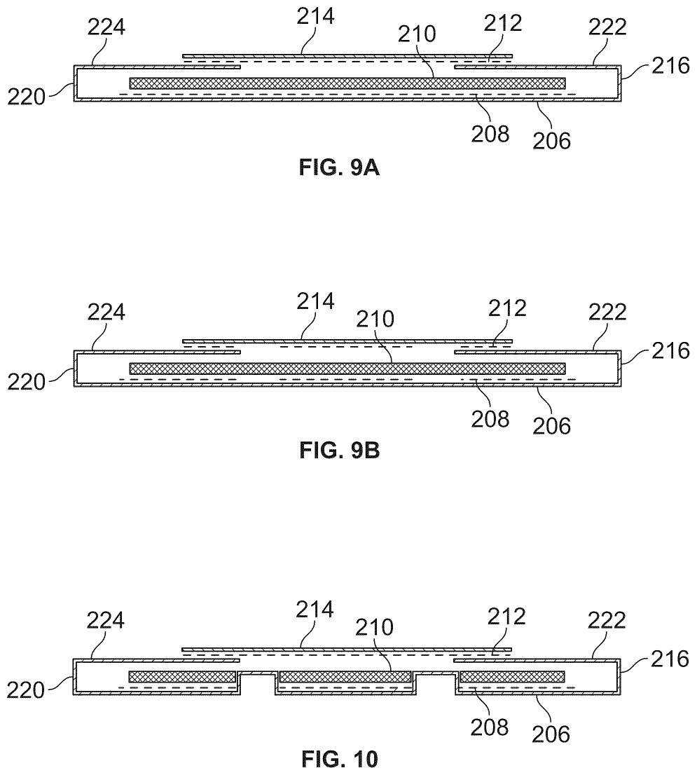

The second, cover web 232 is continuously fed from the source 244, such as a roll of material, to the combining station 250. The cover web 232 may be fed around a series of rollers as it proceeds from the source 244 to the combining station 250. The second adhesive application station 248 is provided between the source 244 and the combining station 250 and applies the second adhesive layer 212 to the first surface of the cover web 232. The second adhesive layer 212 may be applied to the entire first surface of the cover web 232 as shown in FIG. 9A, or may be applied to the first surface of the cover web 232 except to the sections where the channels 204 will be formed as shown in FIG. 9B.

In the combining station 250, the second adhesive layer 212 is brought into contact with the folded over sections 222, 224 and the cover web 232 overlaps the core material layer 210 therebetween. The cover web 232 bonds to the folded over sections 222, 224 and to the core material layer 210 in the combining station 250. This forms a third assembly.

Thereafter, the third assembly passes through the debulking and channel cutting station (shown as debulking and channel cutting station 258a, but can be a debulking and channel cutting station identical to that of debulking and channel cutting station 158b) to form a fourth assembly, and the cutting unit 274 to form the individual inserts 202. Thereafter, the individual inserts 202 are moved to another apparatus (not shown) for completion of the disposable product in which the inserts 202 are encapsulated between a back sheet formed of an impermeable material and at least one hydrophilic top sheet. Other layers may be provided in the final disposable product.

Adhesive layers 208 and 212 may be applied using spray coating or slot coating techniques. In the embodiment shown in FIG. 9B, since the adhesive layers 208, 212 are not applied to the webs 230, 232 where the channel(s) 204 will eventually be die cut by the channel die drum, this assists in preventing the contamination of the channel die drum by adhesive.

FIG. 10 shows an alternative cross-section of an embodiment before the die cutting takes place. In the embodiment used to form the embodiments of FIGS. 9A and 9B, the rotating drum of the core former 236 has a smooth outer surface. This forms a generally uniform thickness of the core material layer 210 on the form-on web 230. The rotating drum 242 of the core former 236 can have structure, for example insert(s) or projection(s) located in its forming surface that inhibit suction at select portions on the forming surface to lessen the thickness of the core material layer 210 on the form-on web 230, or to prevent the core material layer 210 from accumulating on the form-on web 230 above those structures. When no core material layer 210 is deposited on the form-on web 230, when the third assembly is die cut to form the channels 204 therein, only the thickness of the form-on and cover webs 230, 232 are die cut (which web(s) 230, 232 are cut depends upon where the channels 204 are cut).

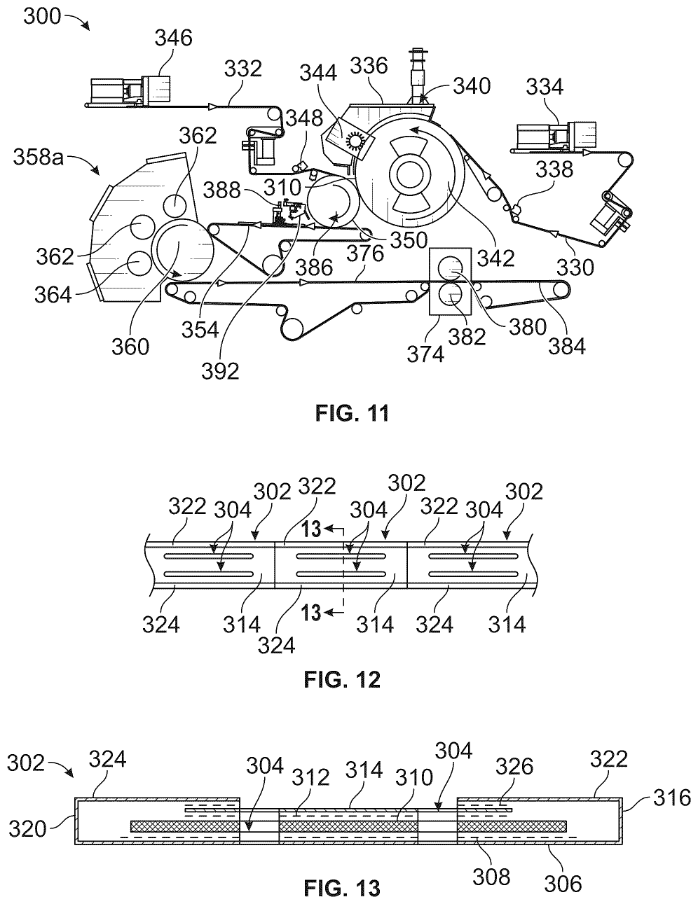

An embodiment of the apparatus 300 which forms the third embodiment of the insert 302 is shown in FIG. 11. Like elements of this embodiment to those of the first and second embodiments are denoted with like reference numerals in the three hundreds.

The first assembly is formed in an identical manner, and therefore the specifics are not repeated herein. The first assembly exits the core former 336 and passes around roller 386 which forms part of the combining station 350 in combination with the vacuum drum 342 of the core former 336. The roller 386 may also be in contact with the conveyor 354.

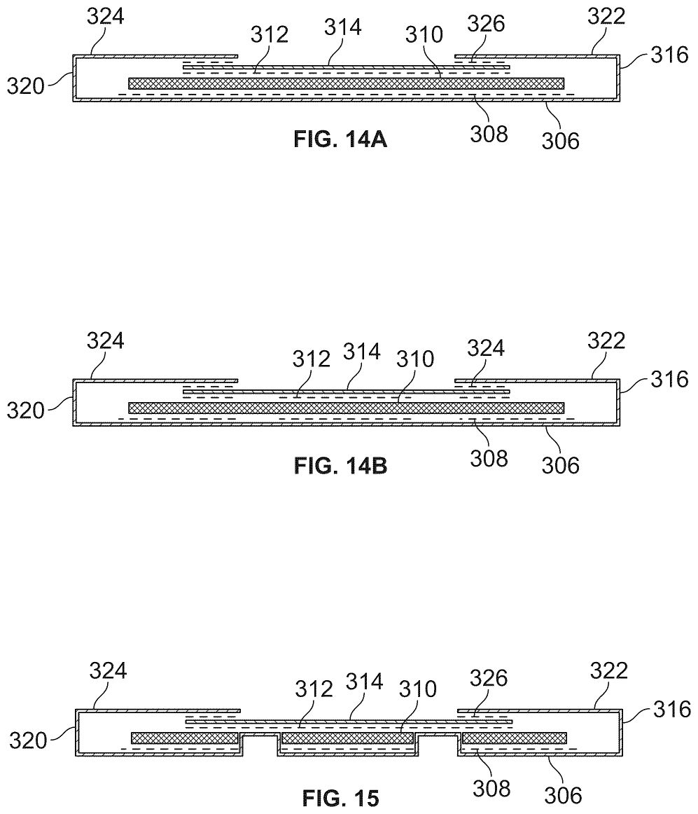

The second, cover web 332 is continuously fed from the source 344, such as a roll of material, to the combining station 350 which is downstream of the core former 336. The cover web 332 may be fed around a series of rollers as it proceeds from the source 344 to the combining station 350. The second adhesive application station 348 is provided between the source 344 and the combining station 350 and applies the second adhesive layer 312 to the first surface of the cover web 332. The second adhesive layer 312 may be applied to the entire first surface of the cover web 332 as shown in FIG. 14A, or may be applied to the first surface of the cover web 332 except to the sections where the channels 304 will be formed as shown in FIG. 14B.

In the combining station 350, the second adhesive layer 312 is brought into contact with, and bonds to, the core material layer 310. The cover web 332 has a width which is the substantially the same as, or less than the core material layer 310. This forms a second assembly.

After combining, the second assembly is moved by the conveyor 354 to a third adhesive application unit 392 provided between the roller 352 and the folding unit 388. The third adhesive application unit 392 applies the third adhesive layer 326 to first and second edge portions of the second surface of the cover web 332. Adhesive is not applied to a central section of the cover web 332. Thereafter, the second assembly passes through the folding unit 388, which may be a folding board, plate, rods, rollers, plow, air curtain or other known folding technologies. In the folding unit 388, the edge portions 316, 320 are folded over onto the cover web 332 to form the folded over sections 322, 324. The folded over sections 322, 324 may partially overlap the second surface of the cover web 332 such that a central portion of the cover web 332 is not covered. This forms a third assembly.

Thereafter, the third assembly passes through the debulking and channel cutting station (shown as debulking and channel cutting station 358a, but can be a debulking and channel cutting station identical to that of debulking and channel cutting station 158b) to form a fourth assembly, and the cutting unit 374 to form the individual inserts 302. Thereafter, the individual inserts 302 are moved to another apparatus (not shown) for completion of the disposable product in which the inserts 302 are encapsulated between a back sheet formed of an impermeable material and at least one hydrophilic top sheet. Other layers may be provided in the final disposable product.

Adhesive layers 308, 312, 326 may be applied using spray coating or slot coating techniques. In the embodiment shown in FIG. 14B, since the adhesive layers 308, 312, 326 are not applied to the webs 330, 332 where the channel(s) 304 will eventually be die cut by the channel die drum, this assists in preventing the contamination of the channel die drum by adhesive.

FIG. 15 shows an alternative cross-section of an embodiment before the die cutting takes place. In the embodiments used to form the embodiments of FIGS. 14A and 14B, the rotating drum 342 of the core former 336 has a smooth outer surface. This forms a generally uniform thickness of the core material layer 310 on the form-on web 330. The rotating drum 342 of the core former 336 can have structure, for example insert(s) or projection(s) located in its forming surface that inhibit suction at select portions on the forming surface to lessen the thickness of the core material layer 310 on the form-on web 330, or to prevent the core material layer 310 from accumulating on the form-on web 330 above those structures. When no core material layer 310 is deposited on the form-on web 330, when the third assembly is die cut to form the channels 304 therein, only the thickness of the form-on web 330 and/or cover web 332 are die cut (which web(s) 330, 332 are cut depends upon where the channels 304 are cut).

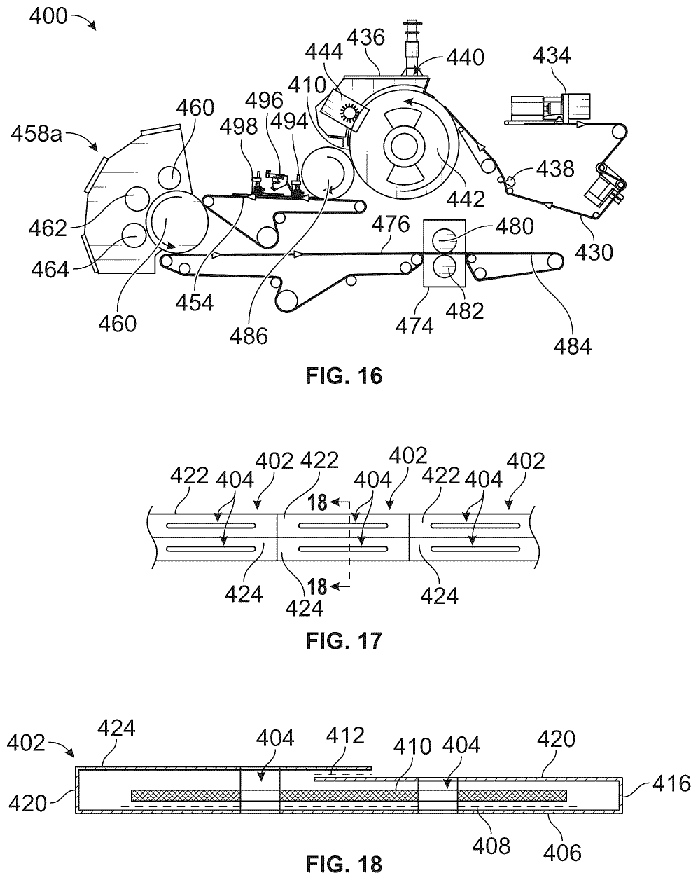

An embodiment of the apparatus 400 which forms the fourth embodiment of the insert 402 is shown in FIG. 16. Like elements of this embodiment to those of the first, second and third embodiments are denoted with like reference numerals in the four hundreds.

The first assembly is formed in an identical manner, and therefore the specifics are not repeated herein. The first assembly exits the core former 436 and passes around roller 486 which may also be in contact with the conveyor 454. The first assembly translates along the conveyor 454 to a first folding unit 494, which may be a folding board, plate, rods, rollers, plow, air curtain or other known folding technologies. The first longitudinally extending edge portion 416 is folded over onto the core material layer 410 to form a first folded over section 422. The folded first assembly then translates along the conveyor 454 to a second adhesive application station 496 at which adhesive is applied to an end portion of the first surface of the first folded over section 422. The first assembly then translates along the conveyor 454 to a second folding unit 498, which may be a folding board, plate, rods, rollers, plow, air curtain or other known folding technologies. The second longitudinally extending edge portion 420 is folded over onto the core material layer 410 and onto the adhesive on the end portion of the first folded over section 422 to form a second folded over section 424. This forms a second assembly.

Thereafter, the second assembly passes through the debulking and channel cutting station (shown as debulking and channel cutting station 458a, but can be a debulking and channel cutting station identical to that of debulking and channel cutting station 158b) to form a third assembly, and the cutting unit 474 to form the individual inserts 402. Thereafter, the individual inserts 402 are moved to another apparatus (not shown) for completion of the disposable product in which the inserts 402 are encapsulated between a back sheet formed of an impermeable material and at least one hydrophilic top sheet. Other layers may be provided in the final disposable product.

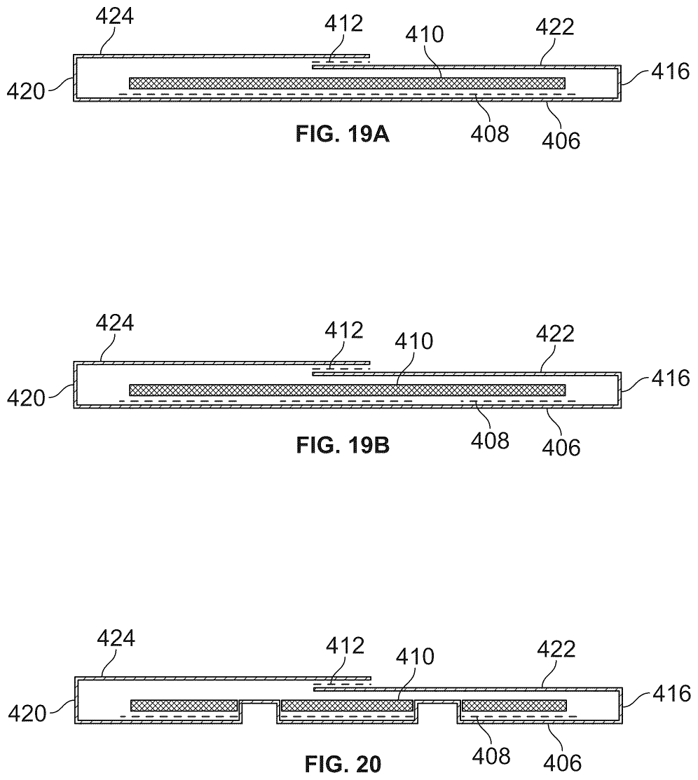

Adhesive layers 408, 412 may be applied using spray coating or slot coating techniques. In the embodiment shown in FIG. 19B, since the adhesive layers 408, 412 are not applied to the web 430 where the channel(s) 404 will eventually be die cut by the channel die drum, this assists in preventing the contamination of the channel die drum by adhesive.

FIG. 20 shows an alternative cross-section of an embodiment before the die cutting takes place. In the embodiments used to form the embodiments of FIGS. 19A and 19B, the rotating drum 442 of the core former 436 has a smooth outer surface. This forms a generally uniform thickness of the core material layer 410 on the web 430. The rotating drum 442 of the core former 436 can have structure, for example insert(s) or projection(s) located in its forming surface that inhibit suction at select portions on the forming surface to lessen the thickness of the core material layer 410 on the web 430, or to prevent the core material layer 410 from accumulating on the web 430 above those structures. When no core material layer 410 is deposited on the web 430, when the second assembly is die cut to form the channels 404 therein, only the thickness of the web 430 is die cut.

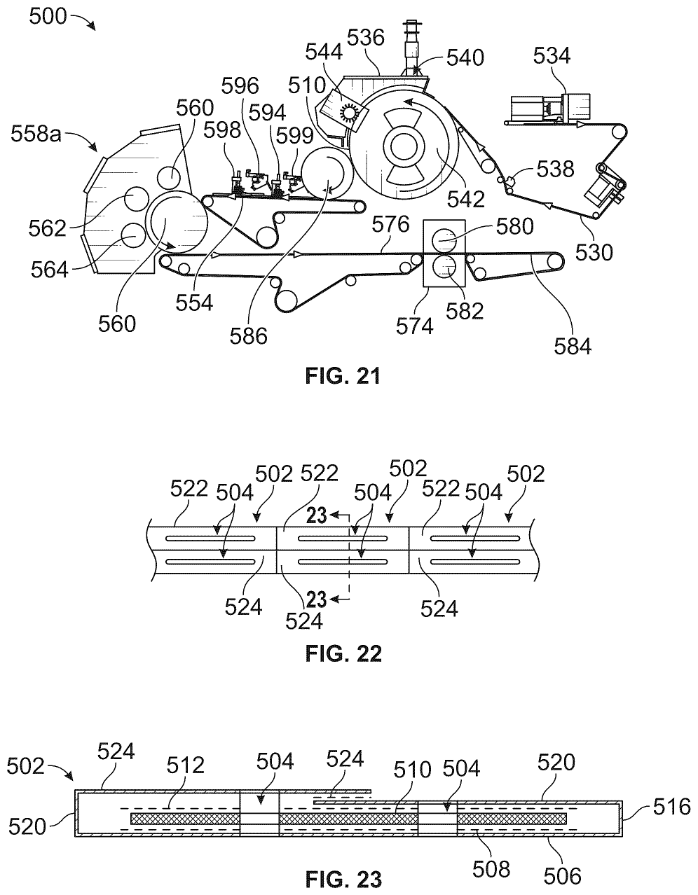

An embodiment of the apparatus 500 which forms the fifth embodiment of the insert 502 is shown in FIG. 21. Like elements of this embodiment to those of the prior embodiments are denoted with like reference numerals in the five hundreds. This embodiment of the apparatus 500 is identical to that of the fourth embodiment of the apparatus 400 except that a third adhesive application station 499 is provided between roller 586 and first folding unit 594.

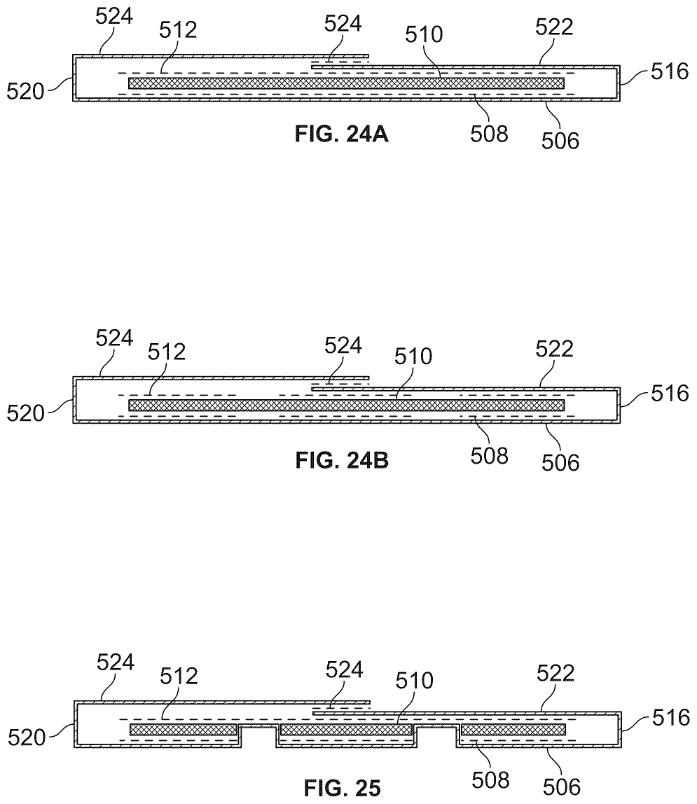

The first assembly exits the core former 536 and passes around roller 586 which may also be in contact with the conveyor 554. The first assembly translates along the conveyor 554 to an optional adhesive application station 599 which deposits a layer of adhesive 512 on the core material layer 510. The adhesive layer 512 may be applied to the entire first surface of the core material layer 510 as shown in FIG. 24A, or may be applied to the core material layer 510 except to the sections where the channels 504 will be formed as shown in FIG. 24B. Thereafter, this assembly translates along the conveyor 554 to the first folding unit 594, which may be a folding board, plate, rods, rollers, plow, air curtain or other known folding technologies. The first longitudinally extending edge portion 516 is folded over onto the adhesive 512 on the core material layer 510 to form a first folded over section 522 and to bond the first folded over section 522 to the core material layer 510. The folded first assembly then translates along the conveyor 554 to a second adhesive application station 596 at which adhesive is applied to an end portion of the first surface of the first folded over section 522. The first assembly then translates along the conveyor 554 to a second folding unit 598, which may be a folding board, plate, rods, rollers, plow, air curtain or other known folding technologies. The second longitudinally extending edge portion 520 is folded over onto the adhesive 512 on the core material layer 510 and onto the adhesive on the end portion of the first folded over section 522 to form a second folded over section 524. This forms a second assembly.

Thereafter, the second assembly passes through the debulking and channel cutting station (shown as debulking and channel cutting station 558a, but can be a debulking and channel cutting station identical to that of debulking and channel cutting station 158b) to form a third assembly, and the cutting unit 574 to form the individual inserts 502. Thereafter, the individual inserts 502 are moved to another apparatus (not shown) for completion of the disposable product in which the inserts 502 are encapsulated between a back sheet formed of an impermeable material and at least one hydrophilic top sheet. Other layers may be provided in the final disposable product.

Adhesive layers 508, 512, 528 may be applied using spray coating or slot coating techniques. In the embodiment shown in FIG. 24B, since the adhesive layers 508, 512, 528 are not applied to the web 530 where the channel(s) 504 will eventually be die cut by the channel die drum, this assists in preventing the contamination of the channel die drum by adhesive.

FIG. 25 shows an alternative cross-section of an embodiment before the die cutting takes place. In the embodiments used to form the embodiments of FIGS. 24A and 24B, the rotating drum 542 of the core former 536 has a smooth outer surface. This forms a generally uniform thickness of the core material layer 510 on the web 530. The rotating drum 542 of the core former 536 can have structure, for example insert(s) or projection(s) located in its forming surface that inhibit suction at select portions on the forming surface to lessen the thickness of the core material layer 510 on the web 530, or to prevent the core material layer 510 from accumulating on the web 530 above those structures. When no core material layer 510 is deposited on the web 530, when the second assembly is die cut to form the channels 504 therein, only the thickness of the web 530 is die cut.

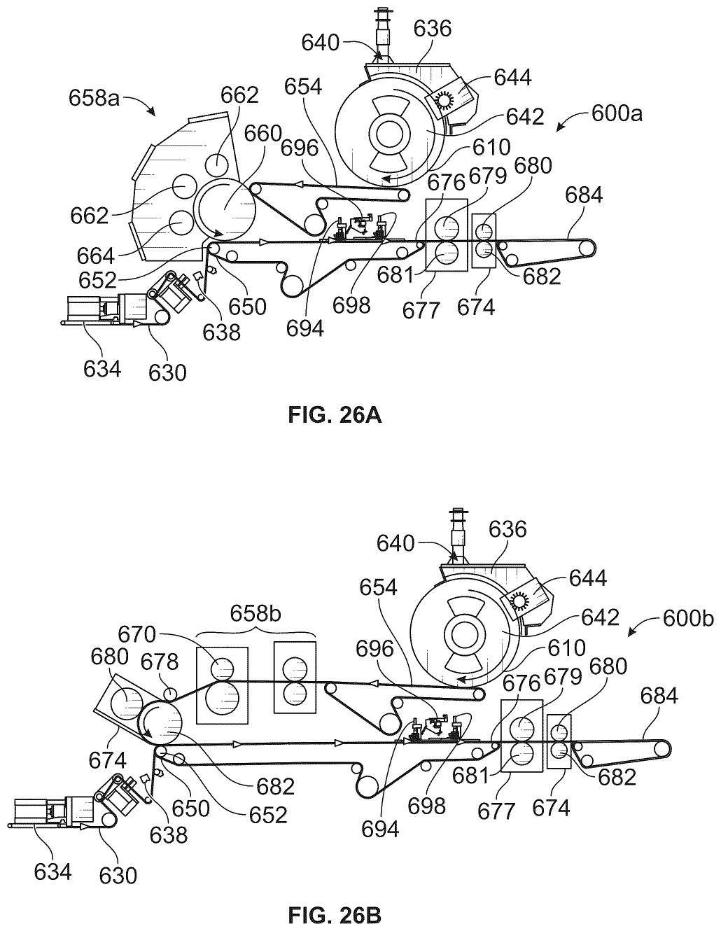

Embodiments of the apparatus 600a, 600b that form the sixth embodiment of the insert 602 is shown in FIGS. 26A and 26B. Like elements of this embodiment to those of the first through fifth embodiments are denoted with like reference numerals in the six hundreds.

In the illustrated embodiment, a super absorbent polymer (SAP)/pulp mixture, or pulp/SAP blend, or pulp only, or SAP only (herein “the core material layer 610”) is fed directly onto the forming surface of the core former 636. The vacuum of the vacuum drum 642 is drawn through holes in the vacuum drum 642 and holds the core material layer 610 against the vacuum drum 642. The vacuum drum 642 may include a continuous three-dimensional fluff receiving pocket or depression or a plurality of discrete, three-dimensional fluff receiving pockets or depressions on an outer circumferential surface thereof as described herein with regard to the other embodiments. The core former 636 may include a scarf device 644 downstream of the outlet 640 which scarfs the core material layer 610 to level the top thereof. In an alternative embodiment, core former 636 may be configured to form the core material layer 610 on a screen.

As shown in FIG. 26A, the core material layer 610 exits the core former 636 onto a conveyor 654, which translates the core material layer 610 to a debulking and channel cutting station 658a. As shown in FIG. 26B, the core material layer 610 exits the core former 636 onto a conveyor 654, which translates the core material layer 610 to a debulking and channel cutting station 658b. Since the core material layer 610 is unwrapped in this embodiment, the channel die drum 684 functions to cut one or more channels 604 through the thickness of the core material layer 610 only.

The die cut the core material layer 610 exits the debulking and channel cutting station 658a or 658bb and is joined to a web 630 at a combining station 650 to form a first assembly. The web 630 is continuously fed from the source 634, such as a roll of material, to the combining station 650. In the embodiment shown in FIG. 26A, the combining station 650 includes the anvil roller 660 and a nip roller 652. The nip roller 652 rotates in coordination with the anvil roller 660. In the embodiment shown in FIG. 26B, the combining station 650 includes the anvil roller 182 and a nip roller 652. The nip roller 652 rotates in coordination with the anvil roller 182. The web 630 may be fed around a series of rollers as it proceeds from the source 634 to the combining station 650. An adhesive application station 638 is provided between the source 634 and the combining station 650 and applies the first adhesive layer 608 to the first surface of the web 630 to which the core material layer 610 is bonded.

The first assembly translates along a conveyor 676 to a first folding unit 694, which may be a folding board, plate, rods, rollers, plow, air curtain or other known folding technologies. The first longitudinally extending edge portion 616 is folded over onto the core material layer 610 to form a first folded over section 622. The folded first assembly then translates along the conveyor 676 to a second adhesive application station 696 at which adhesive is applied to an end portion of the first surface of the first folded over section 622. The first assembly then translates along the conveyor 676 to a second folding unit 698, which may be a folding board, plate, rods, rollers, plow, air curtain or other known folding technologies. The second longitudinally extending edge portion 620 is folded over onto the core material layer 610 and onto the adhesive on the end portion of the first folded over section 622 to form a second folded over section 624. This forms a third assembly.

Thereafter, the third assembly passes through a channel bonding unit 677. In FIGS. 26A and 26B, the channel bonding unit 677 is shown as an ultrasonic bonding unit including an anvil 679 and horn 681 that cooperate to ultrasonically bond facing layers of the web 630 within channels 604. In other embodiments, the channel bonding unit 677 may include components that bond the facing layers of the web 630 at one or more bond points 683 within channels 604 via different mechanical bonding techniques such as, for example, thermal, pressure, or adhesive bonding techniques or various other forms of bonding known in the industry. Thereafter, the third assembly passes through the cutting unit 674 to form the individual inserts 602, which are subsequently moved to another apparatus (not shown) for completion of the disposable product in which the inserts 602 are encapsulated between a back sheet formed of an impermeable material and at least one hydrophilic top sheet. Other layers may be provided in the final disposable product.

Adhesive layers 608, 612 may be applied using spray coating or slot coating techniques.

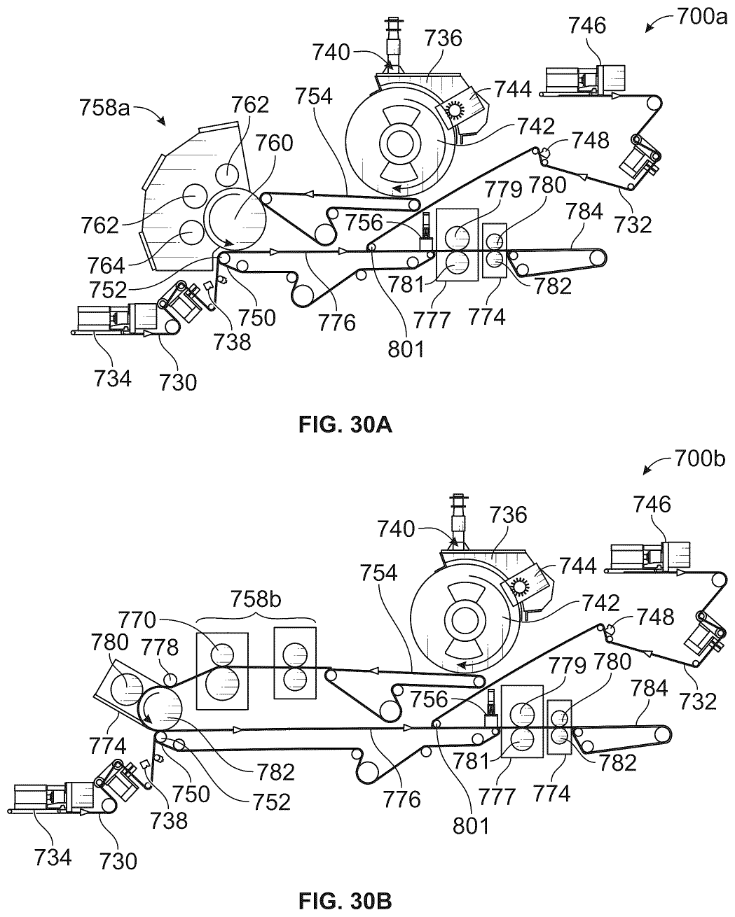

Embodiments of the apparatus 700a, 700b that form the seventh embodiment of the insert 702 is shown in FIGS. 30A and 30B. Like elements of this embodiment to those of the first through sixth embodiments are denoted with like reference numerals in the seven hundreds.

In the illustrated embodiment, a super absorbent polymer (SAP)/pulp mixture, or pulp/SAP blend, or pulp only, or SAP only (herein “the core material layer 710”) is fed directly onto the forming surface of the core former 736. The vacuum of the vacuum drum 760 is drawn through holes in the vacuum drum 760 and holds the core material layer 710 against the vacuum drum 760. The vacuum drum 760 may include a continuous three-dimensional fluff receiving pocket or depression or a plurality of discrete, three-dimensional fluff receiving pockets or depressions on an outer circumferential surface thereof. The core former 736 may include a scarf device 744 downstream of the outlet which scarfs the core material layer 710 to level the top thereof. In an alternative embodiment, core former 736 may be configured to form the core material layer 710 on a screen.

As shown in FIG. 30A, the core material layer 710 exits the core former 736 onto a conveyor 754, which translates the core material layer 710 to a debulking and channel cutting station 758a. As shown in FIG. 30B, the core material layer 710 exits the core former 736 onto a conveyor 754, which translates the core material layer 710 to a debulking and channel cutting station 758b. Since the core material layer 710 is unwrapped in this embodiment, the channel die drum 784 functions to cut one or more channels 704 through the thickness of the core material layer 710 only.

The die cut the core material layer 710 exits the debulking and channel cutting station 758a or 758bb and is joined to a first, form-on web 730 at a combining station 750 to form a first assembly. The form-on web 730 is continuously fed from a source 734, such as a roll of material, to the combining station 750. The form-on web 730 may be fed around a series of rollers as it proceeds from the source 734 to the combining station 750. A first adhesive application station 738 is provided between the source 734 and the combining station 750 and applies the adhesive layer 708 to at least a portion of the first surface of the form-on web 730. Adhesive is not applied to first and second longitudinally extending edge portions 716, 720 of the first surface of the form-on web 730. The adhesive layer 708 may be applied to the entire first surface of the form-on web 730 between the first and second longitudinally extending edge portions 716, 720 as shown in FIG. 4A, or may be applied to the first surface of the form-on web 730 between the first and second longitudinally extending edge portions 716, 720 except to the sections where the channels 704 are formed. In the embodiment shown in FIG. 30A, the combining station 750 includes the anvil roller 782 and a nip roller 752. The nip roller 752 rotates in coordination with the anvil roller 782. In the embodiment shown in FIG. 30B, the combining station 750 includes the anvil roller 182 and a nip roller 752. The nip roller 752 rotates in coordination with the anvil roller 182. The web 730 may be fed around a series of rollers as it proceeds from the source 734 to the combining station 750. An adhesive application station 738 is provided between the source 734 and the combining station 750 and applies the first adhesive layer 708 to the first surface of the web 730 to which the core material layer 710 is bonded.

The first assembly translates along a conveyor 776 and is joined with a second, cover web 732 at a second combining station 801 which may be a roller that is contact with the conveyor 776. The cover web 732 is continuously fed from a source 746, such as a roll of material, to the second combining station 801. The cover web 732 may be fed around a series of rollers as it proceeds from the source 734 to the second combining station 801. A second adhesive application station 748 is provided between the source 746 and the second combining station 801 and applies the adhesive layer 712 the first surface of the cover web 732. The adhesive layer 708 may be applied to the entire first surface of the cover web 732 between the first and second longitudinally extending edge portions 718, 722, or may be applied to the second surface of the cover web 732 between the first and second longitudinally extending edge portions 718, 722 except to the sections where the channels 704 will be formed. This forms a second assembly.

Thereafter, the second assembly passes through an edge bonding unit 756. The edge bonding unit 756 adheres the first longitudinally extending edge portions 716, 718 together by the second adhesive layer 712 and adheres the second longitudinally extending edge portions 720, 722 together by the second adhesive layer 712. The edge bonding unit 756 may be movable roller(s) that press down onto the conveyor 776. Thereafter, the assembly passes through a channel bonding unit 777. In FIGS. 30A and 30B, the channel bonding unit 777 is shown as an ultrasonic bonding unit including an anvil 779 and horn 781 that cooperate to ultrasonically bond facing layers of the web 730 within channels 704. In other embodiments, the channel bonding unit 777 may include components that bond the facing layers of the web 730 at one or more bond points 783 within channels 704 via different bonding techniques such as, for example, thermal, pressure, or adhesive bonding techniques or various other forms of bonding known in the industry. Thereafter, the assembly passes through the cutting unit 774 to form the individual inserts 702, which are subsequently moved to another apparatus (not shown) for completion of the disposable product in which the inserts 702 are encapsulated between a back sheet formed of an impermeable material and at least one hydrophilic top sheet. Other layers may be provided in the final disposable product.

The embodiments shown in FIGS. 30A and 30B can also include folding the form-on web 730 around the core material 710 as shown in the embodiments of FIG. 6 and FIG. 11 to position the cover web 732 on top of the folded over portions of the form-on web 730 or underneath the folded over portions of the form-on web 730.

Adhesive layers 708, 712 may be applied using spray coating or slot coating techniques.

The foregoing is considered as illustrative only of the principles of the disclosure. Furthermore, since numerous modifications and changes will readily occur to those skilled in the art, it is not desired to limit the disclosure to the exact construction and operation shown and described. While the preferred embodiment has been described, the details may be changed without departing from the disclosure, which is defined by the claims.

1. An apparatus configured to make an absorbent insert for an absorbent product, the absorbent insert comprising an absorbent core material positioned between at least one sheet, the apparatus comprising:
a core former configured to provide absorbent core material;
a first adhesive application unit configured to apply a first adhesive to at least a portion of a form-on web of material, wherein the absorbent core material is bonded to the form-on web by the first adhesive; and
a die cutting unit located downstream of the core former and configured to die cut at least one channel that extends through a thickness of at least the absorbent core material.

2. The apparatus of claim 1, wherein the die cutting unit comprises a die and a first roller, the die having a continuous cutting edge which forms the at least one channel and which turns against and in coordination with the first roller.

3. The apparatus of claim 2, further comprising a debulking unit configured to debulk the core material.

4. The apparatus of claim 3, wherein the debulking unit comprises a second roller which turns against and in coordination with the first roller.

5. The apparatus of claim 3, wherein the debulking unit is separate from the die cutting unit.

6. The apparatus of claim 5, further comprising a conveyor between the debulking unit and the die cutting unit.

7. The apparatus of claim 6, wherein the debulking unit comprises a second roller and a third roller.

8. The apparatus of claim 1, wherein the first adhesive application unit does not apply adhesive to a surface of the form-on web where the at least one channel will be die cut.

9. The apparatus of claim 1, wherein the die cutting unit is configured to die cut at least one channel that extends through a thickness of the absorbent core material and the form-on web.

10. The apparatus of claim 1, further comprising at least one folding unit configured to fold the form-on web around a portion of the core material.

11. The apparatus of claim 10, further comprising a second adhesive application unit configured to apply a second adhesive to at least a portion of the form-on web which is folded around the core material.

12. The apparatus of claim 1, further comprising a second adhesive application unit configured to apply a second adhesive to at least a portion of a cover web of material, wherein the absorbent core material is bonded to the cover web by the second adhesive.

13. The apparatus of claim 12, wherein the second adhesive application unit applies does not apply adhesive to a surface of the cover web where the at least one channel will be die cut.

14. The apparatus of claim 12, wherein the die cutting unit is configured to die cut at least one channel that extends through a thickness of the absorbent core material, the form-on web and the cover web.

15. The apparatus of claim 14, further comprising one of an adhesive applicator, an ultrasonic bonding unit, a thermal bonding unit, and a pressure bonding unit located downstream of the die cutting unit and configured to bond the form-on web to the cover web within the channels.

16. The apparatus of claim 1, wherein the at least one channel is formed by the die cutting unit only cutting though the core material.

17. The apparatus of claim 16, further comprising at least one folding unit configured to fold at least one portion of the form-on web around a portion of the core material.

18. The apparatus of claim 17, further comprising one of an adhesive applicator, an ultrasonic bonding unit, a thermal bonding unit, and a pressure bonding unit located downstream of the die cutting unit and configured to bond facing surfaces of the form-on web together within the channels.

19. The apparatus of claim 17, further comprising at least one second adhesive application station downstream of the die cutting unit, wherein the at least one second adhesive application station is configured to deposit adhesive on the form-on web.

20-34. (canceled)
